(19) United States
(12) Patent Application Publication
(10) Pub. No.: US 2008/0311214 A1
(43) Pub. Date: Dec. 18, 2008

(54) Polymerized solid lipid nanoparticles for oral or mucosal delivery of therapeutic proteins and peptides

(75) Inventor: Kollipara Koteswara Rao, Hyderabad (IN)

Correspondence Address: FOLEY AND LARDNER LLP;SUITE 500, 3000 K STREET NW, WASHINGTON, DC, 20007, US

(73) Assignee: Transgene Biotek Limited

(21) Appl. No.: 12/216,313

(22) Filed: Jul. 2, 2008

Related U.S. Application Data
(63) Continuation of application No. 11/397,963, filed on Apr. 5, 2006, now abandoned.

(51) Int. Cl. A61K 9/14; A61K 38/02; A61K 38/28; A61P 3/10; A61K 39/29; A61K 38/21

(52) U.S. Cl. 424/498; 514/2; 514/3; 424/227.1; 424/85.4; 977/906

(57) ABSTRACT

The present invention encompasses lipid nano/micro particles, which have been modified, preferably on their surface, to contain a molecule or ligand, which targets the nano/micro particles to a specific site. The invention also encompasses the use of the modified lipid nano/micro particles for the oral delivery of drugs and antigen delivery systems.

### FIELD OF THE INVENTION

The present invention provides polymerized solid lipid nanoparticles for delivery of drugs, therapeutic protein/peptide, and vaccines. Specifically, the invention provides compositions and methods for treating or preventing disease.

### INTRODUCTION

Although oral administration is the preferred method for administrating medication, many novel therapeutics continue to be administered parenterally. Parenteral administration of drugs or therapeutic proteins/peptides pose many disadvantages, such as patient non-compliance, highly variable bioavailability, and in vitro and in vivo instability. Apart from these problems, parenteral vaccines elicit only humoral immunity and requires repeated injections. For these reasons, there's been great interest in developing an oral system for delivering therapeutic protein/peptide drugs.

Oral vaccines offer the potential to protect against enteric pathogens (by producing localized sIgA), and a wide range of pathogens infecting other mucosa by producing a common disseminated mucosal immune response. Furthermore, oral vaccines may prove particularly useful in the elderly because mucosal immunity, unlike systemic immunity, does not seem to be an age associated dysfunction. Likewise, oral immunization may be beneficial in the very young, because mucosal immunity develops earlier in ontogeny than systemic immunity.

Over the past few decades, various alternative delivery systems were attempted for protein/peptide drugs. The poor intrinsic permeability and high enzymatic degradation in the hostile gastrointestinal environment are the main obstacles for the delivery. Recent advances in biotechnology has led to the development of an increased number of novel therapeutic protein/peptide drugs, but it still remains a challenge to develop a oral delivery system for these therapeutics.

Various formulation approaches, including the use of emulsions, microspheres, nanoparticles, vesicular carriers such as liposomes, permeation enhancers and protease inhibitors, mucoadhesive systems, protein-carrier conjugates and ligand coupled systems, were attempted for oral delivery of protein/peptide drugs. Recently lectin coupled delivery systems were attempted for oral delivery of therapeutic proteins by using M-cell targeting as the mechanism, but lectin has a lower ability to bind particles and therefore, some particles remained intact in the GIT and untargeted(WO6387397). To increase the intensity of targeting, solid lipid nanoparticles made of long chain fatty acids and lipids, in which the intact particle can be taken up via Peyer's patch that resembles the uptake of dietary lipids, having a drug encapsulated within lipid nanoparticles were coated with lectin coupling for M-cell targeting. Receptor mediated bioadhesion of these lectins are also used to convey signals to cells in order to trigger vesicular transport process into or across the polarized epithelial cells. These solid lipid nanoparticles can also cross the intestinal epithelium more effectively than other systems.

Accordingly, the present invention provides polymerized solid lipid nanoparticles comprised of long chain fatty acids and lipids for effective targeting of protein or peptide bioactives to M-cells.

### SUMMARY OF THE INVENTION

In one aspect, the invention provides a polymerized solid lipid nanoparticle system comprising lipids and long chain fatty acids, a therapeutic protein or peptide, an adjuvant, a lectin, at least one polymer, and a pharmaceutically acceptable carrier.

In one embodiment, the therapeutic protein or peptide is selected from the group consisting of gentamycin, Amikacin, insulin, EPO, G-CSF, GM-CSF, Factor VIR, LHRH analogues, Interferons, heparin, Hepatitis ‘B’ surface antigen, typhoid vaccine, and cholera vaccine.

In another embodiment, the pharmaceutically acceptable carrier comprises lectin and drug-loaded lipid particulate carriers. In a further embodiment, the lipid particulate carriers degrade in-vivo and release a therapeutic protein or peptide for a bioactive response. In a further embodiment, the particulate carriers comprise at least one of Beeswax, Behenic acid, caprylic/capric triglyceride, Cetyl palmitate, Cholesterol, Glyceryl trilaurate, Glyceryl trimyristate, Glyceryl tristearate, Glyceryl tripalmitate, Glyceryl monostearate, Glyceryl behenate, hardened fat, monostearate monocitrate glycerol, Propylene glycol palmitic Stearate, mixture of mono, di, tri glycerides of C16-C18 fatty acids, cetyl alcohol, solid paraffin, stearic acid, super polystate, Witepsol H5, and Witepsol W 35.

In one embodiment, the long chain fatty acids are selected from the group consisting of myristic, palmitic, stearic, arachidic, behenic, lignoceric, cerolic, caboceric, monlanic, and melissic acids.

In another embodiment, the nanoparticle system is reservoir-type. In a further embodiment, the reservoir-type system is selected from the group consisting of microcapsule, nanocapsule, or multi particulate type.

In another embodiment, the lipid is solid at room temperature and physiological temperature.

In another embodiment, the lectin is selected from the group consisting of Ulex Europeaus Agglutinin I, wheat germ agglutinin, tomato lectin, Con A/Concavalin A, carbohydrates, and Chitosan and its derivatives.

In another embodiment, the adjuvant is selected from the group consisting of emulsifiers, cryoprotectants, charge modifiers, protease inhibitors, and permeation enhancers.

In another embodiment, the nanoparticle system is in the form of a solution, suspension, gel, paste, elixir, viscous colloidal dispersion, tablet, capsule, or oral controlled release substance.

In another aspect, the invention provides a method for treating diabetes, comprising administering a solid lipid nanoparticle comprising lipids and long chain fatty acids, a therapeutic protein or peptide, an adjuvant, a lectin, at least one polymer, and a pharmaceutically acceptable carrier to a patient in need.

In one embodiment, the therapeutic protein is insulin.

In another embodiment, the lectin is selected from the group consisting of Ulex Europeaus Agglutinin I, wheat germ agglutinin, tomato lectin, and Con A/Concavalin A.

In another embodiment, the administration is oral, sublingual, or buccal.

In another aspect, the invention provides a method for immunizing a mammal, comprising administering to said mammal a nanoparticle system comprising lipids and long chain fatty acids, an antigen, an adjuvant, a lectin, at least one polymer, and a pharmaceutically acceptable carrier.

In one embodiment, the lectin is selected from the group consisting of Ulex Europeaus Agglutinin I, wheat germ agglutinin, tomato lectin, Con A/Concavalin A.

In another embodiment, the antigen is selected from Hepatitis B surface antigen, typhoid antigen, and cholera antigen.

In another aspect, the invention provides a biodegradable nanoparticle system for treating a disease, wherein said nanoparticle system comprises lipids and long chain fatty acids, an therapeutic protein or peptide, an adjuvant, a lectin, at least one polymer, and a pharmaceutically acceptable carrier.

In one embodiment, the therapeutic protein is selected from the group consisting of insulin, EPO, G-CSF, GM-CSF, Factor VIR, LHRH analogues, and Interferons.

### DETAILED DESCRIPTION OF THE INVENTION

The present invention is based on the discovery that the polymerized lipid nanoparticulate system confers enhanced stability against the harsh environment of the gastrointestinal tract and high binding affinity for the mucosal cells of the Peyer's patch. Accordingly, the invention provides an optimal system for administering drugs, therapeutic proteins/peptides, and vaccines. The present invention uses terms and phrases that are well known to those practicing the art. Unless defined otherwise, all technical and scientific terms used herein have the same meaning as commonly understood by one of ordinary skill in the art to which this invention belongs.

Oral delivery of peptide and proteins has proven an elusive target for the pharmaceutical industry over the past few decades. In the present invention, model therapeutic proteins, insulin and vaccine hepatitis ‘B’ vaccine, were selected as model proteins. The conjugation of various peptides and proteins to different polymers and particulate systems has been shown to facilitate the in vitro and in vivo transport of protein moieties across the epithelial cells of the intestine. In this invention different biodegradable and physiologically acceptable particulate systems made up of different lipids and waxes (which include but are not limited to triglycerides, monoglycerides, diglycerides, cetyl palmitate, and bees wax), saturated and unsaturated fatty acids (which include but are not limited to stearic acid, palmitic acid, and oleic acid) were prepared with loading of bioactives (insulin, Hepatitis B vaccine). These bioactive loaded lipid nano/micro particles were coated with carbohydrates or lectins. Examples of the lectins that may be used to modify the lipid nano/micro particles of present invention include but not limited to, lectins specific for fucosyl glycoconjugates, such as Ulex Europeaus Agglutinin I (UAE I); lectins specific for galactose/N-acetylgalactoseamine, such as wheat germ agglutinin (WGA), tomato lectin (Lycopersicum esculentum), lectins specific for mannose/glucose such as con A/concavalin A. In some cases loading step was altered wherein the therapeutic moiety was added during the preparation of lipid nano/micro particles which were later lectinized or the therapeutic moiety was added after the preparation of the lipid nano/micro particles. In-vitro studies showed that insulin was protected from degradation by gastric enzymes. These systems, of different sizes, were fed to diabetic rats, whose blood glucose levels were monitored over time. The pharmacological activity and bioavailability of these systems were shown not only by oral bioactivity but also by the evidence of controlled release of these biopharmaceuticals. Thus the above carrier systems and various strategies mentioned herein, upon further testing and optimization make per oral protein delivery into a reality. The present invention relates to the field of pharmaceutical preparation of traditional injectable drugs given parenterally, peptide and protein pharmaceuticals including vaccines, particularly in the field of such pharmaceutical preparations, which are suitable for oral delivery.

Bioactive Agent encompasses any fluid, composition, or substance that produces a local or systemic therapeutic effect in an animal. The term includes active substances that affect a biological function of an animal directly or as a result of a metabolic or chemical modification associated with the organism or its vicinal environment. For example, a bioactive agent may include any pharmaceutical substance, such as a drug, which may be given to alter a physiological condition of an animal, such as a disease. A bioactive agent is meant to include any type of drug, medication, medicament, vitamin, nutritional supplement, or other compound that is designed to affect a biological function of an animal. The term includes any substance intended for use in the diagnosis or therapeutic treatment or prevention of disease.

For example, bioactive agents may be selected from drugs traditionally administered exclusively by parenteral routes, such as gentamycin and Amikacin, therapeutic peptides and proteins, such as insulin, EPO, G-CSF, GM-CSF, Factor VIR, LHRH analogues and Interferons, other biopharmaceuticals, such as heparin, and vaccines, such as Hepatitis ‘B’ surface antigen, typhoid, and cholera.

Carbohydrates are chemical compounds containing oxygen, hydrogen, and carbon atoms. They consist of monosaccharide sugars of varying chain lengths and that have the general chemical formula Cₙ(H₂O)ₙ or are derivatives of such.

The present invention contemplates conjugating a lipid nanoparticle with a carbohydrate. Suitable carbohydrates include, for example, dextran, Chitosan, and its derivatives which included but not limited to N-carboxymethyl Chitosan and thiolated Chitosan.

Hepatitis B surface antigen is derived from the surface of the Hepatitis B virus and is present in the blood in active Hepatitis B infections. It is also called Australia antigen.

Individual, subject, host, and patient, used interchangeably herein, refer to any mammalian subject for whom diagnosis, treatment, or therapy is desired. In one embodiment, the individual, subject, host, or patient is a human. Other subjects may include, but are not limited to, cattle, horses, dogs, cats, guinea pigs, rabbits, rats, primates, and mice.

Lectin is a sugar-binding protein of non-immune origin that agglutinates cells or precipitates glycoconjugates. Typically, a lectin molecule contains at least two sugar-binding sites because a sugar-binding protein with a single site will not agglutinate or precipitate structures that contain sugar residues Lectins also include sugar-specific enzymes, transport proteins, and toxins if they have, multiple sugar-binding sites. The present invention contemplates numerous lectins, including, for example, Ulex Europeaus Agglutinin I, wheat germ agglutinin, tomato lectin, and Con A/Concavalin A.

Long chain fatty acids generally have straight carbon chains with an even carbon number. Illustrative long chain fatty acids include but are not limited to myristic, palmitic, stearic, arachidic, behenic, lignoceric, cerolic, caboceric, monlanic, and melissic acids.

Solid lipid is a lipid that is solid at room temperature and also at physiological body temperature. A solid lipid is comprised of triglycerides and long chain fatty acids.

Solid lipid nanoparticle system is a nanoparticle comprising lipids and long chain fatty acids, a therapeutic protein or peptide, an adjuvant, a lectin, at least one polymer, and a pharmaceutically acceptable carrier. The inventive solid lipid nanoparticle system may be used to deliver a bioactive agent for treating disease, such as diabetes, or preventing disease, such as Hepatitis B.

Illustrative lipids, long chain fatty acids, and waxes include but are not limited to mono, di, or triglycerides; bees wax; cetyl palmitate; behenic acid; caprylic/capric triglyceride; cholesterol; glyceryl trilaurate; glyceryl trimyristate; glyceryl tristearate; glyceryl tripalmitate; glyceryl monostearate; glyceryl behenate; hardened fat; monostearate monocitrate glycerol; propylene glycol palmitic Stearate; mixture of mono, di, tri glycerides of C16-C18 fatty acids; cetyl alcohol; solid paraffin; stearic acid; super polystate; Witepsol H5; and Witepsol W 35.

Solid lipid nanoparticles are generally spherical in shape and may be administered by oral, sublingual, or buccal routes.

Reservoir-type refers to a nanoparticle system that is composed of microcapsules such that bioactive/drug particles are entrapped within particles.

Therapeutic Protein/polypeptide includes any protein or polypeptide that produces a local or systemic therapeutic effect in an animal. Exemplary therapeutic proteins/polypeptides include but are not limited to gentamycin, Amikacin, insulin, EPO, G-CSF, GM-CSF, Factor VIR, LHRH analogues, Interferons, heparin, Hepatitis ‘B’ surface antigen, typhoid vaccine, and cholera vaccine.

### Lipid Molecules

A variety of ligands can be used to modify the solid polymerized lipid nanoparticles of the present invention in order to target them to a specific cell type. Exemplary ligands, include but are not limited to, carbohydrates, lectins, antibody fragments, and bacterial proteins.

Examples of lectins that may be used to modify the polymerized solid lipid nanoparticles of the present invention, include but are not limited to, lectins specific for fucosyl glycoconjugates, such as Ulex Europeas Agglutinin I (UEA); lectins specific for galactose/n-acetylgalactoseamine, tomato lectin (lycopersicon esculentum), Wheat Germ Agglutinin (WGA); lectins specific for mannose/glucose, such as, con A/concavalan A. These targeting molecules can be derivatized if desired. WO 9503035.

Illustrative carbohydrates include dextran, chitosan and its derivatives which included but not limited to N-carboxymethyl Chitosan and thiolated Chitosan.

In another embodiment of the invention, polymerized solid lipid nanoparticles may be modified with viral proteins or bacterial proteins that have an affinity for a particular residue expressed on a cell surface or that have an affinity for a cell surface protein or receptor. Examples of such proteins include, but are not limited to, cholera toxin b subunit, bacterial adhesotopes.

### Materials to be Loaded

The modified lipid nanoparticles of the present invention find utility in the delivery of vaccines, antigens, allergens, therapeutic agents, and drugs. The polymerized solid lipid nanoparticles of the present invention may be loaded with a variety of bioactive agents or compounds for treating or preventing disease. For example, the nanoparticles may be loaded with therapeutic proteins, chemotherapeutic agents, antibiotics, cytokines, interferon, hormones, antiviral agents, antibacterial agents, antifungal agents, and nucleic acids.

For instance, the inventive nanoparticles may comprise a therapeutic protein for treating a disease, such as diabetes. In another example, the inventive nanoparticles may comprise an antigen for vaccinating against infection, such as hepatitis B.

### Preparation of Solid Lipid Nanoparticles

The polymerized lipid nanoparticles of the present invention may be prepared by a variety of techniques. For example, polymerized solid lipid nanoparticles are prepared by polymerizing the surface of solid lipid nanoparticles prepared by double emulsion or solvent diffusion or other techniques. The polymerization can take place in a solution containing a biologically active substance, such as a drug, protein or antigen, in which case the substance is encapsulated during the polymerization. Alternatively, the solid lipid nanoparticles can be polymerized first, and the biologically active substance can be added later by resuspending the polymerized lipid nanoparticles in a solution of a biologically active substance, and entrapping the substance by sonification of the suspension.

Accordingly, a bioactive agent may be loaded by physically entrapping the particles before, after, or during ligand conjugation; adsorbing the particles; covalently coupling the particles; ionic interaction, or any other means known in the art. Unentrapped biologically active substance can be removed by several means, including repeated centrifugation, decantation, gel filtration, and dialysis. The polymerized solid lipid nanoparticles are then suspended in a buffer solution. The buffer solution has a pH preferably between pH 4.5 and pH 9.5, more preferably at physiological pH.

### Administration of Nanoparticles

The inventive nanoparticles can be administered by any means which optimize uptake by mucosal tissue. For example, oral, sublingual, and buccal may be used. Alternatively, topical, transdermal, and parenteral delivery may also be used. Where oral administration is desired, the inventive nanoparticles can be delivered by tablets, capsules, gels, pastes, elixirs, viscous colloidal dispersions, solutions, suspensions, and oral control release types.

The polymerized lipid nanoparticles of the present invention are suitable for administration to mammals, including humans, as well as other animals and birds. For example, domestic animals such as dogs and cats, as well as domesticated herds, cattle, sheep, pigs and the like may be treated or vaccinated with the polymerized solid lipid nanoparticles of the present invention.

### Vaccine Preparations

Suitable preparations of vaccines include oral vaccinations; injectables, either as liquid solutions or suspensions; solid forms suitable for solution in, suspension in, liquid prior to injection, may also be prepared. The active immunogenic ingredients are often mixed with excipients which are pharmaceutically acceptable and compatible with the active ingredient. Suitable excipients include, for example, water saline, dextrose, glycerol, and combinations thereof. In addition, if desired, the vaccine preparation may also include minor amounts of auxiliary substances such as wetting or emulsifying agents, ph buffering agents, and/or adjuvants which enhance the effectiveness of the vaccine.

Examples of adjuvants which may be used in the present invention, include, but are not limited to: aluminum hydroxide, n-acetyl-muramyl-1-threonyl-d-isoglutamine (thr-mdp), n-acetyl-nor-muramyl-1-alanyl-d-isoglutamine, n-acetylmuramyl-1-alanyl-d-isoglutaminyl-1-alanine-2-(1′-2′-dipalmitoyl-sn-glycero-3-hydroxyphosphoryloxy)-ethylamine.

The vaccines of the invention may be multivalent or univalent. Multivalent vaccines are made from recombinant viruses that direct the expression of more than one antigen.

The vaccine formulations of the invention comprise an effective immunizing amount of the antigenic protein and a pharmaceutically acceptable carrier or excipient. Vaccine preparations comprise an effective immunizing amount of one or more antigens and a pharmaceutically acceptable carrier or excipient. Pharmaceutically acceptable carriers are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration. Effective doses (immunizing amounts) of the vaccines of the invention may also be extrapolated from dose-response curves derived from animal model test systems.

The present invention thus provides a method of immunizing an animal, or treating or preventing various diseases or disorders in an animal, comprising administering to the animal an effective dose of a vaccine encapsulated within lipid nanoparticles of the present invention.

In immunization procedures, the amount of immunogen to be used and the immunization schedule will be determined by a physician skilled in the art and will be administered by reference to the immune response and antibody titers of the subject.

### Kits

The inventive nanoparticles may be presented in a kit comprising one or more unit dosage forms containing an active ingredient for treating or preventing a disease. For example, the present invention contemplates a kit for treating diabetes having a lipid nanoparticle system comprising a solid lipid nanoparticle, a therapeutic protein, and a lectin or a carbohydrate. Additionally, the present invention contemplates a kit for immunizing against hepatitis B having a lipid nanoparticle system comprising a solid lipid nanoparticle, a hepatitis B surface antigen, and a lectin or a carbohydrate.

Specific examples are presented below of methods and constructs for increasing recombinant protein expression in a plant. They are meant to be exemplary and not as limitations on the present invention.

### EXAMPLE 1

### Method for Preparing Insulin Loaded Solid Lipid Nanoparticles

Insulin solution (1-2 mg in 200 μl of 0.01N HCL) was added to 1-2 ml of dichloromethane solution containing 100 mg-200 mg of stearic acid/palmitic acid and 0.5 to 1% lecithin. This mixture was dispersed with an ultrasonic probe for 20-30 sec at 35% amplitude to give W/O primary emulsion (4-6° C.). A double emulsion was formed after addition of 20-50 ml of 1% PVA (Poly vinyl alcohol) to the previous W/O emulsion followed by homogenization at 22,000 rpm for 2-3 min. in ice bath (4-6° C.). This double emulsion was sonicated at 35% amplitude for 2 min in ice bath. Then the solvent was evaporated for 6 hrs under stirring. The insulin-loaded nanoparticles were isolated from the non-encapsulated insulin by ultra centrifugation at 85000×g. They are washed with water for three times to remove any traces of PVA. Finally re-suspended in water and lyophilized using 20-30% of Trehalose as cryoprotectant.

### EXAMPLE 2

### Coupling of Lectin to Insulin Loaded Solid Lipid Nanoparticles

The obtained insulin loaded nanoparticles (100 mg) were dispersed in 1 ml of deionised water and mixed thoroughly with 1-3 ml of 50 mg/ml NHS aqueous solution and stirred for 2 hrs at room temperature. Three to five micro grams of WGA/UEA was dissolved in 500 μl of deionised water and EDAC (30-50 mg) was dissolved in 1 ml deionised water. Both solutions were added to reaction mixture and stirred at 4° C. for 20 hrs. This reaction mixture was centrifuged at 50000 rpm and 4° C. for 15 min. The sediment was lyophilized and supernatant was analyzed for unbound ligand.

The following are the typical examples given for illustration purposes only and do not limit the scope of the invention.

F-1: WGA Conjugated Insulin Loaded Stearic Acid Nanoparticles

Stearic acid—100-200 mg

Insulin—1-2 mg

Lecithin—0.5-1%

Polyvinyl alcohol (1%)—20-50 ml

Trehalose—30%

Wheat Germ Agglutinin (WGA)—3-5 mg.

F-2: UAE Conjugated Insulin Loaded Stearic Acid Nanoparticles

Stearic acid—100-200 mg

Insulin—1-2 mg

Lecithin—0.5-1%

Polyvinyl alcohol (1%)—20-50 ml

Trehalose—30%

Ulex Europeaus Agglutinin (UAE )—3-5 mg.

F-3: WGA Conjugated Insulin Loaded Palmitic Acid Nanoparticles

Palmitic acid—100-200 mg

Insulin—1-2 mg

Lecithin—0.5-1%

Polyvinyl alcohol (1%)—20-50 ml

Trehalose—30%

Wheat Germ Agglutinin (WGA)—3-5 mg.

F-4: UAE Conjugated Insulin Loaded Palmitic Acid Nanoparticles

Palmitic acid—100-200 mg

Insulin—1-2 mg

Lecithin—0.5-1%

Polyvinyl alcohol (1%)—20-50 ml

Trehalose—30%

Ulex Europeaus Agglutinin (UAE)—3-5 mg.

### EXAMPLE 3

### Method for Preparing HBsAg Loaded Solid Lipid Nanoparticles

One -two hundred micro liters of HBsAg solution (60-80 μg of HBsAg) were added to 1-2 ml of dichloromethane solution containing 100mg-200 mg of stearic acid/palmitic acid and 0.5 to 1% lecithin. This mixture was dispersed with an ultra sonic probe for 20-30 sec at 35% amplitude to give W/O primary emulsion (4-6° C.). A double emulsion was formed after addition of 20-50 ml of 1% PVA (Poly vinyl alcohol) to the previous W/O emulsion followed by homogenization at 22,000 rpm for 2-3 min. in ice bath (4-6° C.) The double emulsion was sonicated at 35% amplitude for 2 min in ice bath. Then the solvent was evaporated for 6 hrs under stirring. This HBsAg loaded nanoparticles were isolated from the non-encapsulated insulin by ultra centrifugation at 85000×g. They are washed with water for three times to remove any traces of PVA. Finally re-suspended in water and lyophilized using 20-30% Trehalose as cryoprotectant.

### EXAMPLE 4

### Coupling of Lectin to HBsAg Loaded Solid Lipid Nanoparticles

The obtained HBsAg loaded nanoparticles (100 mg) were dispersed in 1 ml of deionised water and mixed thoroughly with 1-3 ml of 50 mg/ml NHS aqueous solution and stirred for 2 hrs at room temperature. Three to five micro grams of WGA/UEA was dissolved in 500 μl of deionised water and EDAC (30-50 mg) was dissolved in 1 ml deionised water. Both solutions were added to reaction mixture and stirred at 4° C. for 20 hrs. This reaction mixture was centrifuged at 50000 rpm and 4° C. for 15 min. The sediment was lyophilized and supernatant was analyzed for unbound ligand.

The following are the typical examples of the formulations given for illustration and do not limit the scope of the present invention:

F-5: WGA Conjugated HBsAg Loaded Stearic Acid Nanoparticles

Stearic acid—100-200 mg

HBsAg—60-80 micro grams

Lecithin—0.5-1%

Polyvinyl alcohol (1%)—20-50 ml

Trehalose—30%

Wheat Germ Agglutinin (WGA)—3-5 mg.

F-6: UAE Conjugated HBsAg Loaded Stearic Acid Nanoparticles

Stearic acid—100-200 mg

HBsAg—60-80 micro grams

Lecithin—0.5-1%

Polyvinyl alcohol (1%)—20-50 ml

Trehalose—30%

Ulex Europeaus Agglutinin (UAE)—3-5 mg.

F-7: WGA Conjugated HBsAg Loaded Palmitic Acid Nanoparticles

Palmitic acid—100-200 mg

HBsAg—60-80 micro grams

Lecithin—0.5-1%

Polyvinyl alcohol (1%)—20-50 ml

Trehalose—30%

Wheat Germ Agglutinin (WGA)—3-5 mg.

F-8: UAE Conjugated HBsAg Loaded Palmitic Acid Nanoparticles

Palmitic acid—100-200 mg

HBsAg—60-80 micro grams

Lecithin—0.5-1%

Polyvinyl alcohol (1%)—20-50 ml

Trehalose—30%

Ulex Europeaus Agglutinin (UAE)—3-5 mg.

### EXAMPLE 5

### In Vivo Assessment of Ligand Coupled Insulin Loaded Solid Lipid Nanoparticles

Wistar rats (150-200 g) of both sexes were obtained from National Institute of Nutrition, Hyderabad. The animals were housed at a room temperature of 22±2° C. with 12 h light/dark cycle and 45-50% relative humidity. The animals had ad libitum access to a standard chow diet (Nutrilab, Banglore) and water except, wherever indicated. After randomization into various groups, the rats were acclimatized for a period of seven days in the new environment before initiation of the experiment.

Diabetes was induced by streptozotocin (STZ) injection. STZ in rats leads to the development of a clinical syndrome characterized by hyperglycemia, excessive osmotic diuresis, and loss of weight, which is similar to human diabetes. Moreover, the STZ-diabetic rat develops the usual chronic microvascular complications (nephropathy, peripheral and autonomic neuropathy) as observed in diabetic patients. Schaan, B. D., et al. Braz J Med Biol Res, 37:12:1895-1902 (2004).

Animals were made diabetic by injecting 50-60 mg/kg STZ dissolved in sodium citrate buffer, pH 4.5. Rats were fasted overnight before STZ administration. STZ-treated rats were used only when they developed elevated plasma glucose levels (confirmed by qualitative measurements of blood glucose >300 mg% 48 h after injection). Metabolic control was evaluated on the basis of plasma glucose and animal weight at the end of the experiments.

The diabetic rats were randomly divided in to six groups consisting of 4 animals. The lyophilized lectin coupled insulin loaded solid lipid nanoparticle formulations (F1, F2, F3 and F4), mentioned in the above examples, containing 10 IU/Kg of total insulin were administered orally to the respective groups after re-dispersing in the appropriate volume of physiological saline solution. The fifth group was treated with 10 IU/Kg of insulin via subcutaneous route and the sixth group was the control group. The control diabetic rats were not treated with insulin or any drug, but an equivalent volume of physiological saline solution was given orally.

The blood samples were collected at each scheduled time intervals. The plasma was separated by centrifugation and serum glucose levels were estimated as described in Bergmeyer, H. U. Methods of Enzymatic Analysis New York: Academic (1974). Plasma samples of all animals were stored at −80° C. until analyzed.

As show in Table 1,

### EXAMPLE 6

### In Vivo Assessment of Ligand Coupled HBsAg Loaded Solid Lipid Nanoparticles

Male BALB/C mice, aged 6-8 weeks and weighing 25±5 g were procured from National Institute of Nutrition, Hyderabad. The animals were housed at a room temperature of 22±2° C. with 12 h light/dark cycle and 45-50% relative humidity. The animals had ad libitum access to a standard chow diet (Nutrilab, Banglore) and water except, wherever indicated. After randomization into various groups, the mice were accommodated for a period of one week in the new environment before initiation of the experiment.

### Immunization Protocol:

Immediately before administration, the required dose of lyophilized NPs conjugates (containing 10 μg of HBsAg/schedule) was weighed and re-suspended in appropriate volume of physiological saline (0.1 ml). All mice were fasted 6 hr before and 6 hr post dose administration but allowed free access to water.

### Primary Immunization:

The mice were randomly divided in to six groups containing 4 animals each. Four groups of each were orally immunized on two consecutive days with a total of 10 μg of HBsAg entrapped in the above mentioned HBsAg loaded polymerized solid lipid nanoparticulate systems. Similarly other two groups were administered with 10 μg HBsAg by oral solution for two consecutive days.

### Secondary Immunization:

Four weeks after primary immunization, all the groups were immunized with equal booster dose for 2-3 consecutive days as described above.

### Collection of Biological Samples:

From all mice, Plasma and salivary samples were taken at 0 day, 4^{th}, 8^{th} and 16^{th} week of immunizations. Blood from mice were collected from retro orbital plexus under light ether anesthesia. Secretion of saliva was induced by intra peritoneal injection of carbamyl-choline chloride (2 μg/mice), and was collected with capillary tubes. Blood and saliva samples were immediately clarified by centrifugation at 5000 rpm/5 min. The samples were preserved with phenyl methyl sulfonyl fluoride (2 μg), Fetal calf serum (2 μg) and sodium azide (2 μg) as protease inhibitor, alternative substrate for protease activity and preservative, respectively. Saliva and serum samples were stored at −80° C. until analysis. There is a group where in a Hepatitis B was given by conventional intra muscular route.

Antibody titers were measured by ELISA assay using HBsAg as coating antigen and biotin conjugated anti-mouse antibodies to reveal anti-Hobs antibody binding. As shown in Table 2, the inventive lipid nanoparticle may be used to immunize against Hepatitis B.

TABLE 1
Percentage reduction in blood glucose levels after per oral administration of
insulin loaded polymerized solid lipid nanoparticles to STZ induced diabetic rats

| | | Mean % Reduction in serum Glucose Levels | | | | | |
|---|---|---|---|---|---|---|---|
| S. | Time | | | | | Unencapsulated | |
| No. | (hrs) | F-1 | F-2 | F-3 | F-4 | Insulin | Control |
| 1. | 2.0 | 8.1 ± 1.6 | 7.4 ± 1.2 | 8.2 ± 1.0 | 7.2 ± 2.3 | 8.2 ± 2.3 | 1.1 ± 1.2 |
| 2. | 3.0 | 10.2 ± 1.9 | 8.6 ± 1.6 | 9.1 ± 1.8 | 8.4 ± 1.9 | 15.1 ± 1.2 | 2.0 ± 0.6 |
| 3. | 4.0 | 16.2 ± 2.1 | 14.3 ± 2.1 | 12.3 ± 3.2 | 13.5 ± 1.6 | 23.2 ± 3.2 | 1.0 ± 1.2 |
| 4. | 5.0 | 20.1 ± 2.6 | 17.2 ± 3.1 | 18.3 ± 2.4 | 16.5 ± 3.2 | 36.2 ± 1.2 | 0.8 ± 0.5 |
| 5. | 6.0 | 24.3 ± 2.1 | 21.2 ± 2.1 | 20.6 ± 1.5 | 19.8 ± 4.1 | 12.1 ± 4.3 | −2.3 ± 1.9 |
| 6. | 8.0 | 27.2 ± 1.6 | 24.1 ± 1.8 | 24.8 ± 2.6 | 23.6 ± 1.6 | 19.2 ± 3.3 | −2.1 ± 0.5 |
| 7. | 10.0 | 21.3 ± 1.5 | 20.2 ± 1.6 | 20.4 ± 2.1 | 18.9 ± 2.1 | 25.1 ± 3.2 | 1.0 ± 0.9 |
| 8. | 12.0 | 28.2 ± 1.5 | 25.2 ± 1.7 | 23.1 ± 3.1 | 21.2 ± 2.6 | 29.2 ± 1.2 | 1.5 ± 2.9 |

TABLE 2: Salivary and plasma Antibody Titers after oral immunization with polymerized solid lipid nanoparticles & unencapsulated HBsAg to Balb/C mice

| | | Mean Antibody Titre | | |
|---|---|---|---|---|
| | | 4th week | | 8th week |
| S. No. | Formulation | Salivary | Plasma | Salivary |
| 1 | Unencapsulated | 191.33 | 54 | 341.66 |
| | HBsAg | | | |
| 2 | F-5 | 122 | 34 | 467.66 |
| 3 | F-6 | 118.2 | 46 | 410.6 |
| 4 | F-7 | 129.6 | 36 | 472.3 |
| 5 | F-8 | 131.2 | 51 | 423.5 |

What is claimed is:

1. A polymerized solid lipid nanoparticle system comprising lipids and long chain fatty acids, a therapeutic protein or peptide, an adjuvant, a lectin, at least one polymer, and a pharmaceutically acceptable carrier.

2. The nanoparticle system of claim 1, wherein said therapeutic protein or peptide is selected from the group consisting of gentamycin, Amikacin, insulin, EPO, G-CSF, GM-CSF, Factor VIR, LHRH analogues, Interferons, heparin, Hepatitis ‘B’ surface antigen, typhoid vaccine, and cholera vaccine.

3. The nanoparticle system of claim 1, wherein said pharmaceutically acceptable carrier comprises lectin and drug-loaded lipid particulate carriers.

4. The nanoparticle system of claim 3, wherein said lipid particulate carriers degrade in-vivo and release a therapeutic protein or peptide for a bioactive response.

5. The nanoparticle system of claim 3, wherein said particulate carriers comprise at least one of Beeswax, Behenic acid, caprylic/capric triglyceride, Cetyl palmitate, Cholesterol, Glyceryl trilaurate, Glyceryl trimyristate, Glyceryl tristearate, Glyceryl tripalmitate, Glyceryl monostearate, Glyceryl behenate, hardened fat, monostearate monocitrate glycerol, Propylene glycol palmitic Stearate, mixture of mono, di, tri glycerides of C16-C18 fatty acids, cetyl alcohol, solid paraffin, stearic acid, super polystate, Witepsol H5, and Witepsol W 35.

6. The nanoparticle system of claim 1, wherein said long chain fatty acids are selected from the group consisting of myristic, palmitic, stearic, arachidic, behenic, lignoceric, cerolic, caboceric, monlanic, and melissic acids.

7. The nanoparticle system of claim 1, wherein said system is reservoir-type.

8. The nanoparticle system of claim 7, wherein said system is selected from the group consisting of microcapsule, nanocapsule, or multi particulate type.

9. The nanoparticle system of claim 1, wherein said lipid is solid at room temperature and physiological temperature.

10. The nanoparticle system of claim 1, wherein said lectin is selected from the group consisting of Ulex Europeaus Agglutinin I, wheat germ agglutinin, tomato lectin, Con A/Concavalin A, carbohydrates, and Chitosan and its derivatives.

11. The nanoparticle system of claim 1, wherein said adjuvant is selected from the group consisting of emulsifiers, cryoprotectants, charge modifiers, protease inhibitors, and permeation enhancers.

12. The nanoparticle system of claim 1, wherein said system is in the form of a solution, suspension, gel, paste, elixir, viscous colloidal dispersion, tablet, capsule, or oral controlled release substance.

13. A method for treating diabetes, comprising administering a solid lipid nanoparticle comprising lipids and long chain fatty acids, a therapeutic protein or peptide, an adjuvant, a lectin, at least one polymer, and a pharmaceutically acceptable carrier to a patient in need.

14. The method of claim 13, wherein said therapeutic protein is insulin.

15. The method of claim 13, wherein said lectin is selected from the group consisting of Ulex Europeaus Agglutinin I, wheat germ agglutinin, tomato lectin, and Con A/Concavalin A.

16. The method of claim 13, wherein administration is oral, sublingual, or buccal.

17. A method for immunizing a mammal, comprising administering to said mammal a nanoparticle system comprising lipids and long chain fatty acids, an antigen, an adjuvant, a lectin, at least one polymer, and a pharmaceutically acceptable carrier.

18. The method of claim 17, wherein said lectin is selected from the group consisting of Ulex Europeaus Agglutinin I, wheat germ agglutinin, tomato lectin, Con A/Concavalin A.

19. The method of claim 17, wherein said antigen is selected from Hepatitis B surface antigen, typhoid antigen, and cholera antigen.

20. A biodegradable nanoparticle system for treating a disease, wherein said nanoparticle system comprises lipids and long chain fatty acids, an therapeutic protein or peptide, an adjuvant, a lectin, at least one polymer, and a pharmaceutically acceptable carrier.

21. The nanoparticle system of claim 20, wherein said therapeutic protein is selected from the group consisting of insulin, EPO, G-CSF, GM-CSF, Factor VIR, LHRH analogues, and Interferons.
